(19) United States
(12) Patent Application Publication
(10) Pub. No.: US 2015/0005264 A1
(43) Pub. Date: Jan. 1, 2015

(54) PREDICTIVE BIOMARKER FOR HYPOXIA-ACTIVATED PRODRUG THERAPY

(71) Applicant: THRESHOLD PHARMACEUTICALS, INC., South San Francisco, CA (US)

(72) Inventor: Charles Hart, South San Francisco, CA (US)

(21) Appl. No.: 14/375,417

(22) PCT Filed: Jan. 30, 2013
(86) PCT No.: PCT/US2013/023921

§ 371 Date: Jul. 29, 2014

Related U.S. Application Data
(60) Provisional application No. 61/593,249, filed on Jan. 31, 2012.

(51) Int. Cl. C12Q 1/68; G01N 33/574

(52) U.S. Cl. CPC C12Q 1/6886; G01N 33/57496; G01N 2333/988; C12Q 2600/158
USPC 514/94

(57) ABSTRACT

CA-IX levels are predictive of the probability that a cancer patient will respond favorably to cancer therapy involving administration of a hypoxia-activated prodrug. In a first aspect, the present invention provides a method for treating cancer comprising the steps of measuring CA-IX levels in a sample isolated from the patient, and administering a hypoxia-activated prodrug only if the CA-IX level measured is equal to or greater than about 30 pg/mL (e.g. 28.8 pg/mL) CA-IX protein in a serum sample, as may be measured, for example or without limitation, using an ELISA. In one embodiment, a HAP is administered if the measured CA-IX level is equal to or greater than about 75 pg/mL (e.g. 77.1 pg/mL) protein in a serum sample. Thus, in one embodiment, the CA-IX level is measured based on the amount of CA-IX protein in a serum sample.

### CROSS REFERENCE TO RELATED APPLICATION

This application claims the benefit under 35 U.S.C. §119(e) of U.S. Provisional Application Ser. No. 61/593,249 filed Jan. 31, 2012, which is hereby incorporated by reference into this application in its entirety.

### FIELD OF INVENTION

Provided herein are methods related to screening and/or treating cancer patients, based on their CA-IX level profile, with hypoxia activated prodrugs.

### BACKGROUND OF THE INVENTION

Cancer is one of the major causes of human morbidity and mortality. Cancer treatment is challenging because it is difficult to kill cancer cells without damaging or killing normal cells. Damaging or killing normal cells during cancer treatment causes adverse side effects in patients and can limit the amount of anticancer drug administered to a cancer patient. It is also difficult to kill cancer cells in regions distant from the vasculature where anticancer drugs fail to penetrate.

Many cancer cells are more hypoxic relative to normal cells. Tumor hypoxia is associated with resistance to anticancer therapies, cancer relapse, and poor prognosis. Certain drugs in preclinical and clinical development target hypoxic cancer cells. These drugs, called hypoxia-activated prodrugs or “HAPs” are administered in an inactive, or prodrug, form but are activated, and become toxic, in a hypoxic environment. PCT Pat. Pub. Nos. WO 07/002931 and WO 08/083101, each of which is incorporated herein by reference, describe HAPs such as those having a structure defined by Formula I, below:

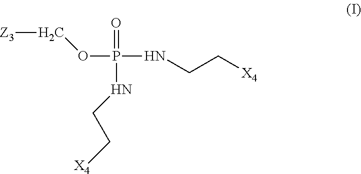

where Z₃ is selected from the group consisting of:

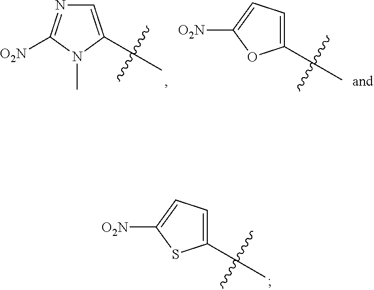

and
X₄ is Cl or Br. The compounds known as TH-302 and TH-281 are particularly promising therapeutic candidates. TH-302, known by the chemical name (2-bromoethyl)({[(2-bromoethyl)amino][(2-nitro-3-methylimidazol-4-yl)methoxy]phosphoryl})amine, has the structure represented below:

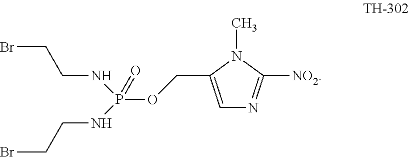 [SMILES: Cn1c(COP(=O)(NCCBr)NCCBr)cnc1[N+](=O)[O-]]

See Duan et al., 2008, “Potent and highly selective hypoxia-activated achiral phosphoramidate mustards as anticancer drugs,” J. Med. Chem. 51: 2412, incorporated herein by reference. Another promising HAP is TH-281, which differs from TH-302 only in that it has 2-chloroethyl groups instead of the 2-bromoethyl groups present in TH-302.

However, while nearly all tumors contain hypoxic regions, there is a wide variability among patients in how hypoxic a tumor of a given cancer type may be. For example, using median tumor pO₂ (mm Hg) as a measure of tumor hypoxia, one study of 33 soft tissue sarcoma patients showed that the median tumor pO₂ ranged from about 1 to about 70 mm Hg (see Nordsmark et al., 2001, Brit. J. Cancer 84(8): 1070-1075). Another study of 58 head and neck cancer patients showed the hypoxic fraction ranged from just above 90% to 1%. Thus, if greater tumor hypoxia correlates with a better response to HAP-mediated anti-cancer therapy, then this variability in tumor hypoxia will translate into a variable response to HAP anti-cancer therapy.

Hypoxia results in a number of biological responses mediated by hypoxia signal transduction pathways. Two of the primary hypoxia signal transduction pathways are the HIF (hypoxia inducible factor) pathway and the UPR (unfolded protein response) pathway. CA-IX (carbonic anhydrase IX) is up-regulated by both of these pathways, and, consistent with studies showing tumor hypoxia generally is a negative prognostic marker, high CA-IX levels have been shown to associate with poor prognosis (see Brit. J. Cancer (2010) 102: 1627-1635; Ann. Surgery (Mar. 2006) 243(3); Brit. J. Cancer (2007) 96:104-109; and Clin. Cancer Res. (2004) 10: 4464-4471).

There remains a need for new methods of determining whether a cancer patient is likely to respond favorably to treatment with HAPs such as TH-302, and/or to treat such patients. The present invention meets these needs.

### SUMMARY OF THE INVENTION

The present invention arises out of the discovery that a cancer patient with high CA-IX level is more likely to respond favorably to HAP anti-cancer therapy than a cancer patient with a lower CA-IX level.

Thus, in a first aspect, the present invention provides a method for treating cancer comprising the steps of measuring CA-IX levels in a sample isolated from the patient, and administering a hypoxia-activated prodrug only if the CA-IX level measured is equal to or greater than about 30 pg/mL (e.g. 28.8 pg/mL) CA-IX protein in a serum sample, as may be measured, for example or without limitation, using an ELISA. In one embodiment, a HAP is administered if the measured CA-IX level is equal to or greater than about 75 pg/mL (e.g. 77.1 pg/mL) protein in a serum sample. Thus, in one embodiment, the CA-IX level is measured based on the amount of CA-IX protein in a serum sample. In another embodiment, the CA-IX level is measured based on the amount of CA-IX RNA in the sample. In one embodiment, a HAP is administered only if the CA-IX level measured is greater than about 150 pg/mL (e.g. 154.2 pg/mL) CA-IX protein in a serum sample. In one embodiment, a HAP is administered only if the CA-IX level measured is greater than about 175 pg/mL (e.g. 178.3 pg/mL) protein in a serum sample.

In other embodiments, the sample is a plasma, whole blood, or pancreatic juice sample or a sample derived from a tumor biopsy, and the CA-IX level is compared to a reference CA-IX level of predetermined value. The reference CA-IX level is determined using a reference population, which may be a population of healthy individuals or a population of cancer patients or a mixed population of the two. The reference CA-IX level, the level at which HAP therapy is indicated for a patient and any others with equal to or higher CA-IX levels, may be, for example and without limitation, the median CA-IX level in a reference population or some multiple of that median, such as two or three times the median CA-IX. The predetermined values provided above (from 30 pg/mL to 175 pg/mL) were obtained using serum samples obtained from cancer patient samples in which CA-IX levels were measured using the ELISA marketed by OncogeneScience (and manufactured by WILEX, Inc., Cambridge Mass.). While similar values would be obtained using other ELISA methods and certain other sample types (blood plasma, for example), any change of sample source or CA-IX assay warrants additional testing to ensure that no adjustment of the predetermined value will improve results based on the different sampling or testing method employed.

In one embodiment, the CA-IX levels are determined using an ELISA. In other embodiments, other methods of measuring CA-IX levels are used. Non-limiting methods for assaying CA-IX include, quantitative western blots, immunohistochemistry (employing CA-IX antibodies) or histochemistry (employing enzyme substrates) of patient samples, including samples derived from tumor biopsies, core biopsies, and needle aspirates; and PET tracers for CA-IX currently in development by Siemens and Wilex. RNA encoding the CA-IX levels can also be used to determine levels in a patient sample. Methods to determine RNA levels are known in the art.

In various embodiments, the HAP administered to the patient is TH-302.

### BRIEF DESCRIPTION OF THE FIGURES

FIGS. 1A, 1B, and 1C show results demonstrating that serum CA-IX is a predictive biomarker for TH-302, as demonstrated by results from thirty melanoma patients undergoing monotherapy with TH-302.

FIG. 2 shows results for the same set of patient data summarized in FIG. 1, graphed using the 2×Median CA-IX level (154.2 pg/mL) as the cut-off for placing patients in the high vs. low CA-IX level groups.

### DETAILED DESCRIPTION OF THE INVENTION

### Definitions

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

“A,” “an,” and, “the” include plural referents unless the context clearly dictates otherwise. Thus, for example, reference to a compound refers to one or more compounds or at least one compound. As such, the terms “a” (or “an”), “one or more”, and “at least one” are used interchangeably herein.

“About” as used herein is used to provide flexibility to a numerical range endpoint by providing that a given value may be “a little above” or “a little below” the endpoint accounting for variations one might see in measurements taken among different instruments, samples, and sample preparations. In one aspect, “about” refers to ±20% of a quantity and includes, but is not limited to, ±15%, ±10%, and ±5% of the quantity.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

“Administering” or “administration of” a drug to a patient (and grammatical equivalents of this phrase) refers to direct administration, which may be administration to a patient by a medical professional or may be self-administration, and/or indirect administration, which may be the act of prescribing a drug. For example, a physician who instructs a patient to self-administer a drug and/or provides a patient with a prescription for a drug is administering the drug to the patient.

“Solid tumor” refers to solid tumors including, but not limited to, metastatic tumors in bone, brain, liver, lungs, lymph node, pancreas, prostate, skin and soft tissue (sarcoma).

“Biomarkers” generally refers to biological molecules, and quantitative and qualitative measurements of the same, that are indicative of a disease state. “Prognostic biomarkers” correlate with disease outcome, independent of therapy. For example, tumor hypoxia is a negative prognostic marker—the higher the tumor hypoxia, the higher the likelihood that the outcome of the disease will be negative. “Predictive biomarkers” indicate whether a patient is likely to respond positively to a particular therapy. For example, HER2 profiling is commonly used in breast cancer patients to determine if those patients are likely to respond to Herceptin® (trastuzumab, Genentech). “Response biomarkers” provide a measure of the response to a therapy and so provide an indication of whether a therapy is working. For example, decreasing levels of prostate specific antigen (PSA) generally indicate that anti-cancer therapy for a prostate cancer patient is working.

“Blood” refers to blood which includes all components of blood circulating in a subject including, but not limited to, red blood cells, white blood cells, plasma, clotting factors, small proteins, platelets and/or cryoprecipitate. This is typically the type of blood which is donated when a human patient gives blood. Plasma is known in the art as the yellow liquid component of blood, in which the blood cells of whole blood are typically suspended. It makes up about 55% of the total blood volume. Blood plasma can be prepared by spinning a tube of fresh blood containing an anti-coagulant in a centrifuge until the blood cells fall to the bottom of the tube. The blood plasma is then poured or drawn off. Blood plasma has a density of approximately 1025 kg/m³, or 1.025 kg/l.

“Cancer” refers to leukemias, lymphomas, carcinomas, and other malignant tumors, including solid tumors, of potentially unlimited growth that can expand locally by invasion and systemically by metastasis. Examples of cancers include, but are not limited to, cancer of the adrenal gland, bone, brain, breast, bronchi, colon and/or rectum, gallbladder, head and neck, kidneys, larynx, liver, lung, neural tissue, pancreas, prostate, parathyroid, skin, stomach, and thyroid. Certain other examples of cancers include, acute and chronic lymphocytic and granulocytic tumors, adenocarcinoma, adenoma, basal cell carcinoma, cervical dysplasia and in situ carcinoma, Ewing's sarcoma, epidermoid carcinomas, giant cell tumor, glioblastoma multiforma, hairy-cell tumor, intestinal ganglioneuroma, hyperplastic corneal nerve tumor, islet cell carcinoma, Kaposi's sarcoma, leiomyoma, leukemias, lymphomas, malignant carcinoid, malignant melanomas, malignant hypercalcemia, marfanoid habitus tumor, medullary carcinoma, metastatic skin carcinoma, mucosal neuroma, myeloma, mycosis fungoides, neuroblastoma, osteo sarcoma, osteogenic and other sarcoma, ovarian tumor, pheochromocytoma, polycythermia vera, primary brain tumor, small-cell lung tumor, squamous cell carcinoma of both ulcerating and papillary type, hyperplasia, seminoma, soft tissue sarcoma, retinoblastoma, rhabdomyosarcoma, renal cell tumor, topical skin lesion, veticulum cell sarcoma, and Wilm's tumor.

“Clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity or side effect.

“Dose” and “dosage” refer to a specific amount of active or therapeutic agents for administration. Such amounts are included in a “dosage form,” which refers to physically discrete units suitable as unitary dosages for human subjects and other mammals, each unit containing a predetermined quantity of active agent calculated to produce the desired onset, tolerability, and therapeutic effects, in association with one or more suitable pharmaceutical excipients such as carriers.

“Having the same cancer” refers to comparing one patient to another or alternatively, one patient population, which may be a reference population, to another patient population. For example, and without limitation, the two patients or patient population will each have or be suffering from melanoma.

“Hypoxia activated prodrug” or “HAP” refers to a prodrug wherein the prodrug is less active or inactive, relative to the corresponding drug, and comprises the drug and one or more bioreducible groups. HAPs include prodrugs that are activated by a variety of reducing agents and reducing enzymes, including without limitation single electron transferring enzymes (such as cytochrome P450 reductases) and two electron transferring (or hydride transferring) enzymes. In some embodiments, HAPs are 2-nitroimidazole triggered hypoxia-activated prodrugs. Examples of HAPs include, without limitation, TH-302, TH-281, PR104 and AQ4N. Methods of synthesizing TH-302 are described in PCT Pat. App. Pub. Nos. WO 07/002931 and WO 08/083101, incorporated herein by reference. Methods of synthesizing PR104 are described in US Pat. App. No. 2007/0032455, incorporated herein by reference. Other examples of HAPs are described, for example, in US Pat. App. Nos. 2005/0256191, 2007/0032455 and 2009/0136521 (each of which is incorporated herein by reference) and PCT Pat. App. Pub. Nos. WO 00/064864, WO 05/087075, and WO 07/002931 (incorporated herein by reference).

“Isolated” refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

“Pancreatic juice sample” refers to pancreatic secretions and isolates of such secretions obtained, e.g., by a physician.

“Patient” and “subject” are used interchangeably to refer to a mammal in need of treatment for cancer. Generally, the patient is a human. Generally, the patient is a human diagnosed with cancer. In certain embodiments a “patient” or “subject” may refer to a non-human mammal used in screening, characterizing, and evaluating drugs and therapies, such as, a non-human primate, a dog, cat, rabbit, pig, mouse or a rat.

A “predetermined value” for CA-IX as used herein, is so chosen that a patient with a level of CA-IX higher than or equal to the predetermined value is likely to experience a more desirable clinical outcome than patients with levels of CA-IX lower than the predetermined value, or vice-versa. Levels of proteins and/or RNA, such as those disclosed in the present invention, are associated with clinical outcomes. One of skill in the art can determine such predetermined values by measuring levels of CA-IX in a patient population to provide a predetermined value. Optionally, a predetermined value for CA-IX level in one patient population can be compared to that from another to optimize the predetermined value to provide a higher predictive value. In various embodiments, a predetermined value refers to value(s) that best separate patients into a group with more desirable clinical outcomes and a group with less desirable clinical outcomes. Such predetermined value(s) can be mathematically or statistically determined with methods well known in the art in view of this disclosure.

“Prodrug” refers to a compound that, after administration, is metabolized or otherwise converted to a biologically active or more active compound (or drug) with respect to at least one property. A prodrug, relative to the drug, is modified chemically in a manner that renders it, relative to the drug, less active or inactive, but the chemical modification is such that the corresponding drug is generated by metabolic or other biological processes after the prodrug is administered. A prodrug may have, relative to the active drug, altered metabolic stability or transport characteristics, fewer side effects or lower toxicity, or improved flavor (for example, see the reference Nogrady, 1985, Medicinal Chemistry A Biochemical Approach, Oxford University Press, New York, pages 388-392, incorporated herein by reference). A prodrug may be synthesized using reactants other than the corresponding drug.

“QnD” or “qnd” refers to drug administration once every n days. For example QD (or qd) refers to once every day or once daily dosing, Q2D (or q2d) refers to a dosing once every two days, Q7D refers to a dosing once every 7 days or once a week, Q5D refers to dosing once every 5 days.

“Reduction” of a symptom or symptoms (and grammatical equivalents of this phrase) refers to decreasing the severity or frequency of the symptom(s), or elimination of the symptom(s).

“Solid tumor” refers to a cancer other than leukemia.

“Suitable for a therapy” or “suitably treated with a therapy” shall mean that the patient is likely to exhibit one or more desirable clinical outcome as compared to patients having the same cancer and receiving the same therapy but possessing a different characteristic that is under consideration for the purpose of the comparison. In one aspect, the characteristic under consideration is a genetic polymorphism or a somatic mutation. In another aspect, the characteristic under consideration is expression level of a gene or a polypeptide. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease free survival. In another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, may exhibit more than one more desirable clinical outcomes as compared to patients having the same cancer and receiving the same therapy but not possessing the characteristic; as defined herein, the patient is considered suitable for the therapy. In another such aspect, a patient possessing a characteristic may exhibit one or more desirable clinical outcome but simultaneously exhibit one or more less desirable clinical outcome. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

“Therapeutically effective amount” of a drug refers to an amount of a drug that, when administered to a patient with cancer, will have the intended therapeutic effect, e.g., alleviation, amelioration, palliation or elimination of one or more manifestations of cancer in the patient. A therapeutic effect does not necessarily occur by administration of one dose, and may occur only after administration of a series of doses. Thus, a therapeutically effective amount may be administered in one or more administrations.

“Treating” or “treatment of” a condition or patient refers to taking steps to obtain beneficial or desired results, including clinical results. For purposes of this invention, beneficial or desired clinical results include, but are not limited to, alleviation or amelioration of one or more symptoms of cancer; diminishment of extent of disease; delay or slowing of disease progression; amelioration, palliation, or stabilization of the disease state; or other beneficial results. Treatment of cancer may, in some cases, result in partial response or stable disease.

“Tumor” refers to an abnormal growth of tissue resulting from uncontrolled, progressive multiplication of cells and serving no or substantially no physiological function. A tumor is also known as a neoplasm.

When a marker, such as CA-IX, is “used as a basis” for identifying or selecting a patient for a treatment described herein, the marker can be measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjustment of dosage; (g) predicting likelihood of clinical benefits; or (h) toxicity. As would be well understood by one in the art, measurement of a biomarker in a clinical setting is a clear indication that this parameter was used as a basis for initiating, continuing, adjusting and/or ceasing administration of the treatments described herein.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Abbreviations used in the description include:
CT—Computed tomography (CT)
D5W—5% dextrose in water
DLT—Dose limiting toxicity
HAP(s)—Hypoxia Activated Prodrug(s)
NSCLC—Non-Small Cell Lung Cancer
PD—Progressive disease
PR—Partial response
RECIST—Response Evaluation Criteria In Solid Tumors
SCLC—Small Cell Lung Cancer
SD—Stable disease
SLD—Sum of the longest diameters
TGD—Tumor growth delay
TGI—Tumor growth inhibition

### DESCRIPTIVE EMBODIMENTS

The disclosure further provides diagnostic, predictive, prognostic and therapeutic methods, which are based, at least in part, on determination of the identity of the expression level of a marker of interest. In particular, the amount of CA-IX in a cancer patient sample can be used to predict whether the patient is likely to respond favorably to cancer therapy utilizing a hypoxia-activated prodrug.

Thus, information obtained using the diagnostic assays described herein is useful for determining if a subject is suitable for cancer treatment of a given type. Based on the predictive information obtained, a doctor can recommend a therapeutic protocol, which may include administration of a hypoxia-activated prodrug, useful for reducing the malignant mass or tumor in the patient or treat cancer in the individual. The information obtained may also be prognostic, in that it can indicated whether a patient has responded favorably or unfavorably to cancer therapy. Generally, if CA-IX levels rise after administration of a cancer therapy, the therapy may not be as efficacious as other therapies, and if CA-IX levels decline after therapy, the therapy is efficacious.

A patient's likely clinical outcome following a clinical procedure such as a therapy or surgery can be expressed in relative terms. For example, a patient having a particular CA-IX expression level who receives HAP therapy may experience relatively longer overall survival than a patient or patients not having the CA-IX expression level who receive HAP therapy. The patient having the particular CA-IX expression level, alternatively, can be considered as likely to survive if administered HAP therapy. Similarly, a patient having a particular expression level who receives HAP therapy may experience relatively longer progression free survival, or time to tumor progression, than a patient or patients not having the CA-IX expression level who receive HAP therapy. The patient having the particular CA-IX expression level, alternatively, can be considered as not likely to suffer tumor progression if administered HAP therapy. Further, a patient not having a particular CA-IX expression level who receives HAP therapy may experience relatively shorter time to tumor recurrence than a patient or patients having the expression level who receive HAP therapy. The patient having the particular CA-IX expression level, alternatively, can be considered as not likely to suffer tumor recurrence if administered HAP therapy. Yet in another example, a patient having a particular expression level if administered HAP therapy may experience relatively more complete response or partial response than a patient or patients not having the genotype or expression level who receive HAP therapy. The patient having the particular genotype or expression level, alternatively, can be considered as likely to respond to HAP therapy. Accordingly, a patient that is likely to survive, or not likely to suffer tumor progression, or not likely to suffer tumor recurrence, or likely to respond following a clinical procedure is considered suitable for the clinical procedure, treatment with a HAP.

It is to be understood that information obtained using the diagnostic assays described herein may be used alone or in combination with other information, such as, but not limited to, expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and the like. In a particular aspect, the expression level can be used in a diagnostic panel each of which contributes to the final diagnosis, prognosis, or treatment selected for a patient.

The present invention arises out of the discovery that a cancer patient with high CA-IX level is more likely to respond favorably to HAP anti-cancer therapy than a cancer patient with a lower CA-IX level. This is remarkable in that high CA-IX levels correlate with a poor prognosis, but it is just these patients that respond best to HAP therapy.

### Diagnostic Methods

Thus, in one aspect, a method is provided for aiding in the selection of or selecting a hypoxia-activated prodrug therapy for a cancer patient, comprising, or alternatively consisting essentially of, or yet further consisting of, determining the CA-IX protein or RNA level in a serum sample isolated from the patient, wherein the hypoxia-activated prodrug therapy is selected for the patient if the level is equal to or exceeds a predetermined level (value) or the hypoxia-activated prodrug therapy is not selected if the level is below the predetermined level (value). In one aspect, the predetermined level is a protein level of about 30 pg/mL CA-IX protein in the serum, or alternatively about 75 pg/mL, or alternatively about 150 pg/mL, or alternatively about 175 pg/mL CA-IX protein in the serum. In one aspect, the therapy is selected for patients exhibiting progression-free survival as compared to similarly situated patients with the marker and did not receive the HAP therapy.

In one aspect, the hypoxia-activated prodrug therapy comprises, or alternatively consists essentially of, or yet further consists of (2-bromoethyl)({[(2-bromoethyl)amino][(2-nitro-3-methylimidazol-4-yl)methoxy]phosphoryl})amine (TH-302) or (2-chloroethyl)({[(2-chloroethyl)amino][(2-nitro-3-methylimidazol-4-yl)methoxy]phosphoryl})amine (TH-281).

In some embodiments, cancer patients that benefit from the diagnostic method include those suffering from various solid tumors, for example and without limitation, hypoxic solid tumors, blood cancers, and the like. In some embodiments, patients that benefit from the diagnostic method include those suffering from various solid tumors and undergoing monotherapy with TH-302.

Any suitable sample can be used for the method. Non-limiting examples of such include one or more of a serum sample, whole blood, pancreatic juice sample or a tumor sample, which can be a isolated from a needle biopsy, core biopsy and needle aspirate.

Any suitable method can be used to measure the CA-IX protein, RNA, or other suitable read-outs for CA-IX levels, examples of which are described herein and/or are well known to the skilled artisan. In some embodiments, determining the level of CA-IX comprises determining the expression of CA-IX, such as, e.g., by determining the CA-IX mRNA or CA-IX protein concentration in a patient sample. To this extent, mRNA of the sample can be isolated, if necessary, after adequate sample preparation steps, e.g. tissue homogenization, and hybridized with marker specific probes, in particular on a microarray platform with or without amplification, or primers for PCR-based detection methods, e.g. PCR extension labeling with probes specific for a portion of the marker mRNA.

In further embodiments, the level of CA-IX is determined by the polypeptide or protein concentration of the CA-IX, e.g. with CA-IX specific ligands, such as antibodies or specific binding partners. The binding event can, e.g., be detected by competitive or non-competitive methods, including the use of labeled ligand or CA-IX specific moieties, e.g. antibodies, or labeled competitive moieties, including a labeled CA-IX standard, which compete with marker proteins for the binding event. If the marker specific ligand is capable of forming a complex with the CA-IX, the complex formation can indicate expression of the CA-IX in the sample.

This disclosure also provides a kit for determining if a hypoxia-activated prodrug therapy is suitable for treatment of a cancer patient, comprising means for determining the serum protein level of a CA-IX protein in a sample isolated from the patient and instructions for use. In a further aspect, the kit further comprises the HAP therapy.

### Therapeutic Methods

Thus, in a first aspect, the present invention provides a method for treating cancer comprising, or alternatively consisting essentially of, or yet further consisting of the steps of measuring CA-IX levels in a sample from said patient, and administering a hypoxia-activated prodrug only if the CA-IX level measured is greater than a predetermined level. In one embodiment, a HAP is administered only if the CA-IX protein level is greater than about 30 pg/mL (e.g. 28.8 pg/mL) in the serum. In one embodiment, a HAP is administered only if the CA-IX protein level measured is greater than about 75 pg/mL (e.g. 77.1 pg/mL) in the serum. In one embodiment, a HAP is administered only if the CA-IX protein level measured is greater than about 150 pg/mL (e.g. 154.2 pg/mL) in the serum. In one embodiment, a HAP is administered only if the CA-IX protein level measured is greater than about 175 pg/mL (e.g. 178.3 pg/mL) in the serum. In various embodiments, the sample is a plasma, serum, whole blood, or pancreatic juice sample.

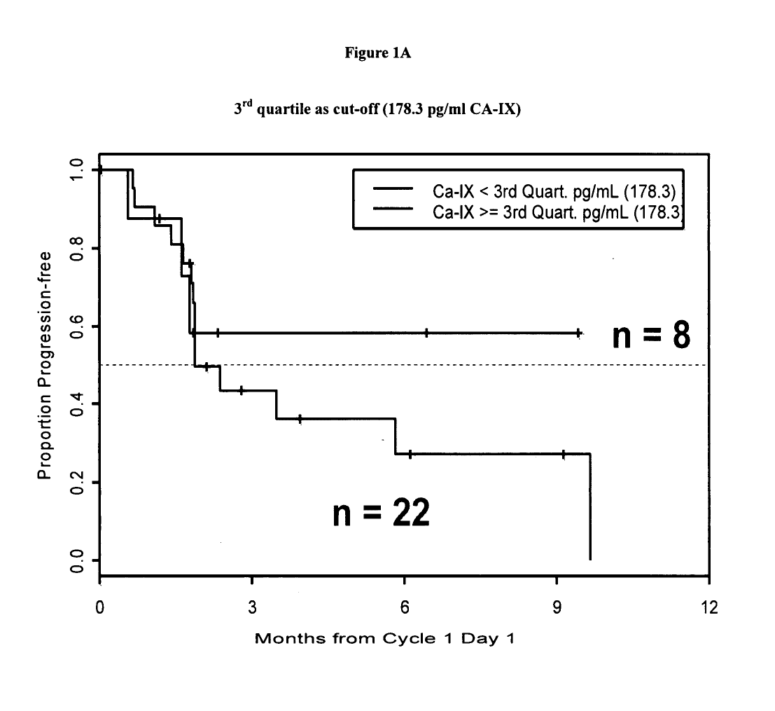

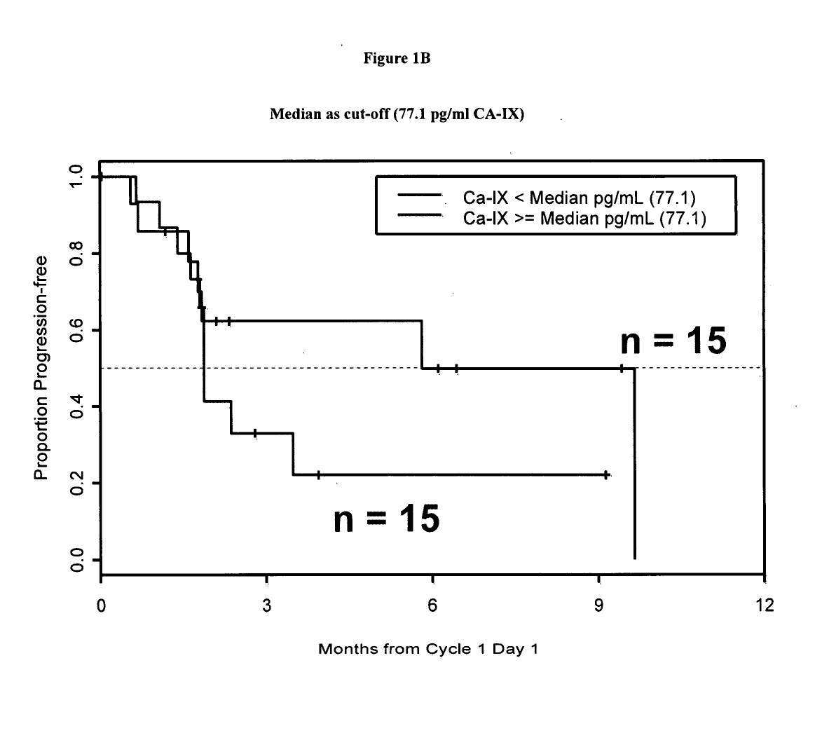

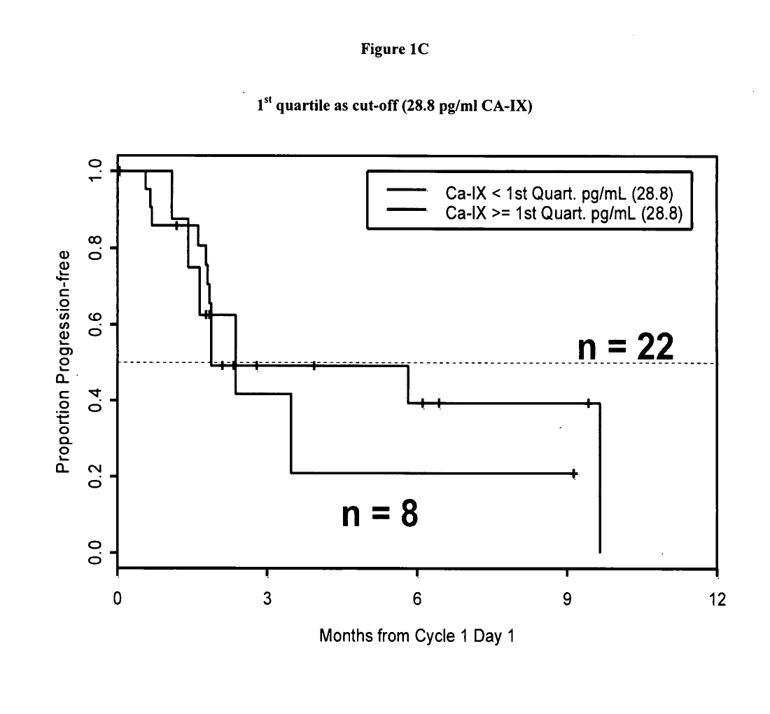

In one important embodiment, the HAP is TH-302. In a clinical trial of TH-302 in melanoma patients, CA-IX levels were measured in serum samples taken from patients prior to TH-302 administration. Measured levels varied widely, with the first quartile having levels of 28.8 pg/mL CA-IX or less, the median being 77.1 pg/mL CA-IX, and the 3^{rd} quartile having 178.3 pg/mL or less. These 25%, 50%, and 75% quartile cut-offs were compared to progression-free survival for the patients whose CA-IX levels were determined, and the results demonstrated that higher CA-IX levels correlated to a better response to TH-302, as shown in FIG. 1.

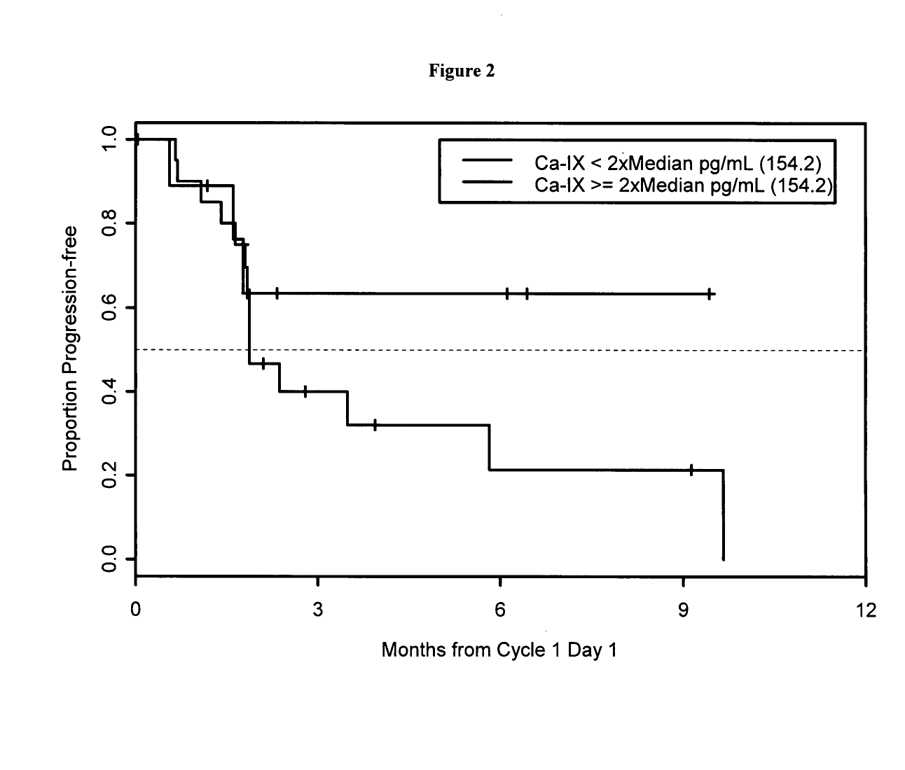

The data set used to generate the graphs in FIG. 1 was used to generate the graph in FIG. 2, which uses the 2×Median value of 154.2 pg/mL as the cut-off for dividing patients into high and low CA-IX level groups. Using this value as the cut-off, one sees an even more dramatic example of the predictive value of CA-IX levels in determining whether a patient will respond to TH-302 or other HAP therapy.

In another embodiment, the hypoxia-activated prodrug comprises (2-chloroethyl)({[(2-chloroethyl)amino][(2-nitro-3-methylimidazol-4-yl)methoxy]phosphoryl})amine (TH-281). In one embodiment, the cancer patient is suffering from melanoma. In one embodiment, patient sample is one or more of a serum sample, a whole blood sample, a pancreatic juice sample, or a tumor sample. In various embodiments, the CA-IX protein level is determined by a method comprising quantitative western blot, ELISA, immunohistochemistry, histochemistry, or use of a PET tracer.

### Methods to Measure or Determine CA-IX Levels

CA-IX levels can be measured in accordance with the methods of the invention by any means known in the art. While CA-IX levels can be readily expressed in pg/mL from serum samples, other measurement units are readily useable in the methods of the invention by those of skill in the art upon contemplation of this disclosure.

In one embodiment, CA-IX levels are determined using an enzyme linked immunosorbent assay (ELISA), including but not limited to the ELISA that can be performed in accordance with and the materials contained in the CA-IX ELISA product marketed by OncogeneScience (and manufactured by WILEX, Inc., Cambridge Mass.). In one embodiment, CA-IX levels are determined using Western blot analysis. In one embodiment, CA-IX levels are determined using solid-phase extraction and matrix-assisted laser desorption/ionization mass spectrometry. In one embodiment, CA-IX levels are determined using surface-enhanced laser desorption/ionization time-of-flight (SELDI-TOF) mass spectrometry. In one embodiment, CA-IX levels are determined using protein arrays based on multiplexing a sandwich-ELISA system with chemiluminescent or fluorescent detection of analytes whose respective capture antibodies are spotted in arrays within each well of a sample plate (e.g. a 96-well microplate).

CA-IX levels from the tissue, serum or liquid sample to be analyzed may easily be detected or isolated using techniques which are well known to one of skill in the art, including but not limited to Western blot analysis. For a detailed explanation of methods for carrying out Western blot analysis, see, e.g., Sambrook and Russell (2001) “Molecular Cloning: A Laboratory Manual,” Third Edition. The protein detection and isolation methods employed herein can also be such as those described in Harlow and Lane, (1999) “Using Antibodies: A Laboratory Manual.” This can be accomplished, for example, by immunofluorescence techniques employing a fluorescently labeled antibody coupled with light microscopic, flow cytometric, or fluorimetric detection. The antibodies (or fragments thereof) useful in the present disclosure may, additionally, be employed histologically, as in immunofluorescence or immunoelectron microscopy, for in situ detection of CA-IX levels. In situ detection may be accomplished by removing a histological specimen from a patient, and applying thereto a labeled antibody. The antibody (or fragment) is preferably applied by overlaying the labeled antibody (or fragment) onto a biological sample. Through the use of such a procedure, it is possible to determine not only the presence of CA-IX levels, but also its distribution in the examined tissue. Using the present disclosure, one of ordinary skill will readily perceive that any of a wide variety of histological methods (such as staining procedures) can be modified in order to achieve such in situ detection.

Moreover, PET imaging can also be used to assess CA-IX levels for purposes of the present invention. WILEX is developing a reagent, REDECTANE® (INN: 1241-Girentuximab), that detects CA-IX by PET and is based on an antibody. The reagent is being developed for the pre-surgical diagnosis of clear cell renal cell cancer. REDECTANE is the radioactively labeled form of the antibody Girentuximab, which binds specifically to Carbonic anhydrase IX (also CA-IX, MN or G250 antigen), which is found on over 90% of clear cell renal cell carcinomas but not on normal renal tissue. SIEMENS is developing another reagent that detects CA-IX, [F18]VM4-037, a radiopharmaceutical consisting of a sulfonamide covalently attached to the positron-emitting isotope fluorine F18 with CA-IX-binding and radioisotopic activities. Upon administration, the sulfonamide moiety of [F18]VM4-037 binds to carbonic anhydrase IX isoenzyme (CA-IX).

For detection of RNA (or mRNA) levels, recognized methods can be utilized. These methods are not limited by the technique that is used to identify the expression level of the RNA of interest. Such methods include nuclease protection assays, northern blots, in situ hybridization, reverse transcriptase Polymerase Chain Reaction (RT-PCR), Real-Time Polymerase Chain Reaction, expressed sequence tag (EST) sequencing, cDNA microarray hybridization or gene chip analysis, statistical analysis of microarrays (SAM), subtractive cloning, Serial Analysis of Gene Expression (SAGE), Massively Parallel Signature Sequencing (MPSS), and Sequencing-By-Synthesis (SBS). See for example, Carulli et al., (1998) J. Cell. Biochem. 72 (S30-31): 286-296; Galante et al., (2007) Bioinformatics, Advance Access (Feb. 3, 2007).

SAGE, MPSS, and SBS are non-array based assays that can determine the expression level of RNAs by measuring the frequency of sequence tags derived from polyadenylated transcripts. SAGE allows for the analysis of overall RNA expression patterns with digital analysis. See for example, Velculescu et al., (1995) Science 270 (5235):484-487; Velculescu (1997) Cell 88 (2):243-251.

MPSS technology allows for analyses of the expression level of virtually all genes in a sample by counting the number of individual mRNA molecules produced from each gene. As with SAGE, MPSS does not require that genes be identified and characterized prior to conducting an experiment. MPSS has a sensitivity that allows for detection of a few molecules of mRNA per cell. Brenner et al. (2000) Nat. Biotechnol. 18:630-634; Reinartz et al., (2002) Brief Funct. Genomic Proteomic 1: 95-104.

SBS allows analysis of gene expression by determining the differential expression of gene products present in sample by detection of nucleotide incorporation during a primer-directed polymerase extension reaction.

SAGE, MPSS, and SBS allow for generation of datasets in a digital format that simplifies management and analysis of the data. The data generated from these analyses can be analyzed using publicly available databases such as Sage Genie (Boon et al., (2002) PNAS 99:11287-92), SAGEmap (Lash et al.,(2000) Genome Res 10:1051-1060), and Automatic Correspondence of Tags and Genes (ACTG) (Galante (2007), supra). The data can also be analyzed using databases constructed using in house computers (Blackshaw et al. (2004) PLoS Biol, 2:E247; Silva et al. (2004) Nucleic Acids Res 32:6104-6110)).

Thus, any of a variety of methods can be used to assess CA-IX levels in a patient or sample taken from the patient for the purpose of predicting whether the patient will respond favorably to hypoxia-activated prodrug therapy. If the CA-IX level in the patient or patient sample is higher than or equal to a predetermined value for the CA-IX level, the patient is administered a HAP therapy, such as TH-302, but if the CA-D(level is below that predetermined value, then the patient is administered an anti-cancer therapy other than HAP therapy.

1. A method for treating cancer in a patient, comprising the steps of measuring a CA-IX protein or RNA level in a sample isolated from said patient and
administering a hypoxia-activated prodrug to said patient if the measured level is equal to or exceeds a predetermined value or administering a cancer therapy other than a therapy comprising administration of a hypoxia-activated prodrug if such measured level does not exceed said predetermined value.

2. The method of claim 1, wherein said predetermined value is about 30 pg/mL of protein in a serum sample.

3. The method of claim 2, wherein said predetermined level is about 75 pg/mL of protein.

4. The method of claim 3, wherein said predetermined level is about 150 pg/mL of protein.

5. The method of claim 4, wherein said predetermined level is about 175 pg/mL of protein.

6. The method of claim 1, wherein the hypoxia-activated prodrug comprises (2-bromoethyl)({[(2-bromoethyl)amino][(2-nitro-3-methylimidazol-4-yl)methoxy]phosphoryl})amine (TH-302) or (2-chloroethyl)({[(2-chloroethyl)amino][(2-nitro-3-methylimidazol-4-yl)methoxy]phosphoryl})amine (TH-281).

7. The method of claim 1, wherein the cancer patient is suffering from melanoma.

8. The method of claim 1, wherein the patient sample is a serum sample, a whole blood sample, a pancreatic juice sample, or a tumor sample.

9. The method of claim 1, wherein the CA-IX protein level is determined by a method comprising ELISA, western blot, immunohistochemistry, histochemistry, or use of a PET tracer.

10. A method for aiding in the selection of or selecting a hypoxia-activated prodrug therapy for a cancer patient, comprising determining the CA-IX protein or RNA level in a sample from the patient, wherein the hypoxia-activated prodrug therapy is selected for the patient if the level is equal to or exceeds a predetermined level or the hypoxia-activated prodrug therapy is not selected if the level is below the predetermined level.

11. The method of claim 10, wherein said predetermined level is about 30 pg/mL of protein in a serum sample.

12. The method of claim 10, wherein said predetermined level is about 75 pg/mL of protein in a serum sample.

13. The method of claim 10, wherein said predetermined level is about 150 pg/mL of protein in a serum sample.

14. The method of claim 10, wherein said predetermined level is about 175 pg/mL of protein in a serum sample.

15. The method of claim 10, wherein the hypoxia-activated prodrug comprises (2-bromoethyl)({[(2-bromoethyl)amino][(2-nitro-3-methylimidazol-4-yl)methoxy]phosphoryl})amine (TH-302) or (2-chloroethyl)({[(2-chloroethyl)amino][(2-nitro-3-methylimidazol-4-yl)methoxy]phosphoryl})amine (TH-281).

16. The method of claim 10, wherein the cancer patient is suffering from melanoma.

17. The method of claim 10, wherein the patient sample is one or more of a serum sample, whole blood, pancreatic juice sample or a tumor sample.

18. The method of claim 10, wherein the CA-IX protein level is determined by a method comprising ELISA, western blot, immunohistochemistry, histochemistry, or use of a PET tracer.

19. A kit for determining if a hypoxia-activated prodrug therapy is suitable for treatment of a cancer patient, comprising means for determining the serum protein level or RNA level of a CA-IX protein in a sample isolated from the patient and instructions for use.

20. The method of claim 6, wherein the cancer patient is suffering from melanoma.
